(19) United States
(12) Patent Application Publication
(10) Pub. No.: US 2014/0081176 A1
(43) Pub. Date: Mar. 20, 2014

(54) SURGICAL TECHNIQUE(S) AND/OR DEVICE(S)

(71) Applicant: Chandra Hassan, Middleburg Heights, OH (US)

(72) Inventor: Chandra Hassan, Middleburg Heights, OH (US)

(21) Appl. No.: 14/076,914

(22) Filed: Nov. 11, 2013

Related U.S. Application Data
(63) Continuation of application No. PCT/US11/31940, filed on Apr. 11, 2011.

(51) Int. Cl. A61B 5/107; A61M 1/00; A61B 19/00; A61B 17/32; A61M 25/10; A61M 25/00; A61B 17/064; A61B 17/072; A61B 5/00; A61B 1/04

(52) U.S. Cl. CPC A61B 5/1076; A61B 5/6853; A61B 5/4836; A61M 1/0023; A61B 19/5202; A61B 1/04; A61M 25/10181; A61M 25/0026; A61B 19/46; A61M 1/0058; A61B 17/0644; A61B 17/072; A61B 17/320016
USPC 600/593; 606/219; 227/180.1

(57) ABSTRACT

One or more techniques and/or devices are disclosed for promoting standardization of the dissection of a portion of a patient's stomach, such as during laparoscopic sleeve gastrectomy (LSG), for example. In this way, LSG can be improved at least by allowing a surgeon and/or patient to choose a post surgery stomach size in a quantifiable manner. For example, a patient could discuss various stomach sizing options with their doctor and select the option believed to be most beneficial to the patient. Moreover, one or more techniques and/or devices are disclosed for decreasing tissue trauma while performing surgical stapling, such as during LSG. In this way, surgical procedures that implement stapling can be improved, as reduced tissue trauma generally accelerates healing time, among other things. For example, staples and/or staple cartridges may be designed to reduce pinching or pressure points along a staple line while achieving desired closure and/or hemostasis.

### RELATED APPLICATION

This application is a continuation of PCT/US11/31940, filed on Apr. 11, 2011, entitled “SURGICAL TECHNIQUE(S) AND/OR DEVICE(S)”, at least some of which may be incorporated herein.

### BACKGROUND

In the field of bariatric surgery, laparoscopic sleeve gastrectomy (LSG) is an emerging procedure commonly accepted as an alternative to gastric bypass, lap banding, and/or bowel re-sectioning surgery as a treatment for morbid obesity, for example. That is, LSG is generally believed to be a quicker, less complex operation, considered easier to perform, while achieving comparable resolution of co-morbidity and weight loss rates. To perform LSG, the stomach may be freed at least some of the blood supply along the greater curvature of the stomach (and possibly other adhesions as well), linear staplers may be introduced to divide the stomach into a tube or sleeve shape, and a dissected portion of the stomach may be removed. Removal of the dissected portion typically reduces the body's capacity to produce ghrelin, which may decrease stimulation of hunger for the patient. Moreover, the pylorus is generally not removed during LSG such that food may remain in the stomach longer, enhancing the ‘full’ feeling for the patient until the food is ultimately passed on. Additionally, bowel rearrangement is generally unnecessary during LSG, thus mitigating marginal ulcers and so forth.

### SUMMARY

This Summary is provided to introduce a selection of concepts in a simplified form which are further described below in the Detailed Description. This Summary is not intended to identify key factors or essential features of the claimed subject matter, nor is it intended to be used to limit the scope of the claimed subject matter.

One or more techniques and/or devices are disclosed to promote standardization of the dissection of a portion of a patient's stomach, such as during laparoscopic sleeve gastrectomy (LSG), for example. In this way, LSG can be improved at least by allowing a surgeon and/or patient to choose a post surgery stomach size in a (more) quantifiable manner. For example, a patient could discuss various stomach sizing options with their doctor and select the option believed to be most beneficial to the patient.

Additionally, one or more techniques and/or devices are disclosed for decreasing tissue trauma while performing surgical stapling, such as during LSG. In this way, surgical procedures that implement stapling can be improved, as reduced tissue trauma generally accelerates healing time, among other things. For example, staples and staple cartridges may be designed to reduce pinching and/or pressure points along a staple line (e.g., reducing the amount and/or degree of tissue that may be crushed during stapling) while achieving desired closure and/or hemostasis.

According to one aspect, a bougie (e.g. a sizing tube) may be introduced prior to performing LSG, in an orogastric fashion which enables a surgeon to (more) accurately size a remaining portion of a patient's stomach and/or remove an excess portion dissected from the stomach, for example. The bougie may take the form of a clear tube, and may comprise markings which indicate one or more sets of standardized units to generally determine a more precise amount of stomach tissue to maintain. Moreover, the tube may be hollow, thus enabling an endoscopic camera and/or a light to be inserted, for example, while the tube is being introduced into the patient or alternatively, at a later time. In one embodiment, the tube may comprise the camera and/or a built in light. According to one aspect the end of the tube may be closed, as will be discussed herein.

According to another aspect, the bougie may comprise an inflatable member (e.g. a balloon) configured to be operatively coupled to and close off the end of the tube inserted into the patient's stomach. Therefore, the balloon may be inflated as gas and/or other substance(s) may be provided through the interior of the hollow tube. In one example, the balloon may be proximal with the interior of the patient's stomach after the device is inserted, for example. Further, the bougie and/or tube may also comprise a means for inflating the balloon. As an example, the tube may comprise one or more channels configured to inflate and/or deflate the balloon. Moreover, additional channels may run along at least a portion of a length of the tube to serve various purposes, as will be discussed herein. For example, one or more of the additional channels may be configured to inflate the stomach, deflate the stomach, and/or apply a hemostat to facilitate blood clotting. A surgeon may insert the sleeve-tube device into the patient's stomach while the balloon is in a deflated state, and inflate the balloon to block the pyloric valve during LSG surgery, for example. In this way, the lower intestines may be separated from the stomach in a secure fashion, thus substantially controlling the flow of fluids, solids, and/or gases between the stomach, the duodenum, and the small intestine (and vice versa). It will be appreciated that the advantages of blocking the pyloric valve will be discussed in greater detail herein.

According to yet another aspect, a surgical stapler may be used to cut and/or divide the stomach into two portions: a remaining portion and a dissected portion. The surgical stapler may also be configured to seal the respective portions by firing staples implanted in the tissue. For example, the surgical stapler may comprise one or more stapler cartridges and a set of surgical staples, both of which may be configured to reduce pressure points and crushing along the staple line relative to conventional surgical staplers. To this end, the stapler cartridges may be assembled in a jaw like manner, and setup to move between at least an open position and a closed position, for example. In one exemplary embodiment, at least one of the stapler cartridges would be arched in a manner which maintains at least a substantial gap between the jaws while the cartridge is in the closed position. It will be appreciated that the gap may be one of a semi-circular shape and/or any other shape (e.g., so as to compress tissue within the gap less than conventional arrangements). Further, the size of the gap and/or the length of a bridge of a staple may be based on the thickness and/or type of tissue being stapled. In another example, the staple cartridge could be configured to provide various staple patterns configured to promote a desired amount of leakage from the stomach and/or a desired number of staples used.

In another yet another embodiment, the surgical staples themselves may be configured to reduce trauma as well. Generally, once conventional staples are implanted, the staples may be deformed in a manner which crushes tissue due to the legs touching and/or bending substantially close to the bridge of the staple. Accordingly, the surgical staples of the present application may be configured to maintain a substantial gap between pointed ends of the legs of the staple and the bridge, for example. Moreover, the size of the gap may be based at least in part on the type of tissue being stapled.

To the accomplishment of the foregoing and related ends, the following description and annexed drawings set forth certain illustrative aspects and implementations. These are indicative of but a few of the various ways in which one or more aspects may be employed. Other aspects, advantages, and novel features of the disclosure will become apparent from the following detailed description when considered in conjunction with the annexed drawings.

### DESCRIPTION OF THE DRAWINGS

The application is illustrated by way of example and not limitation in the figures of the accompanying drawings, in which like references indicate similar elements and in which:

FIGS. 1A-1H are illustrations of a sleeve-tube device in accordance with one or more aspects described herein.

FIG. 2 is an illustration of an example digestive system.

FIG. 3 is an illustration of a sleeve-tube device within an example digestive system in accordance with one or more aspects described herein.

FIG. 4 is a flow diagram illustrating an example embodiment where one or more techniques described herein may be implemented.

FIGS. 5A-5I are illustrations of various views of exemplary staples.

FIGS. 6A-6B are illustrations of an exemplary surgical stapler in accordance with one or more aspects described herein.

FIG. 7A-7H are illustrations of exemplary surgical stapler cartridges in accordance with one or more aspects described herein.

FIG. 8 is an illustration of surgical staple lines within an example digestive system in accordance with one or more aspects described herein.

FIG. 9 is a flow diagram illustrating an example embodiment where one or more techniques described herein may be implemented.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are illustrated in block diagram form in order to facilitate describing the claimed subject matter.

One or more techniques and/or devices are disclosed to promote standardizing dissection of a portion of a patient's stomach. In this way, laparoscopic sleeve gastrectomy (LSG) may be improved, as post surgery stomach sizes may be standardized based on a sizing bougie with standardized markings. According to one aspect, the sizing bougie may comprise an inflatable member operatively coupled to one end of the bougie to facilitate stapling and leakage testing. For example, a doctor might recommend a larger stomach size to a patient so weight loss may be achieved in a more stable fashion. During surgery, it is believed to be helpful for a surgeon to slightly inflate the stomach prior to stapling to facilitate higher quality stapling. Further, once inflated, the balloon may be configured to block adjacent openings to enable proper leakage testing, for example.

Additionally, one or more techniques and/or devices are disclosed for decreasing tissue trauma while performing surgical stapling. In this way, surgical stapling may be improved, as reducing tissue trauma is believed to accelerate healing time, for example. In one embodiment, the staples and staple cartridges may be configured to reduce pinching and/or pressure points along a staple line. In this manner, staples may be configured to promote a desired (e.g., maximize) the gap between points of the legs and the bridge of the staple. Similarly, staple cartridges may be configured to arch in a manner which may reduce surface area of the stapler contacting tissue while the stapler is in the closed position.

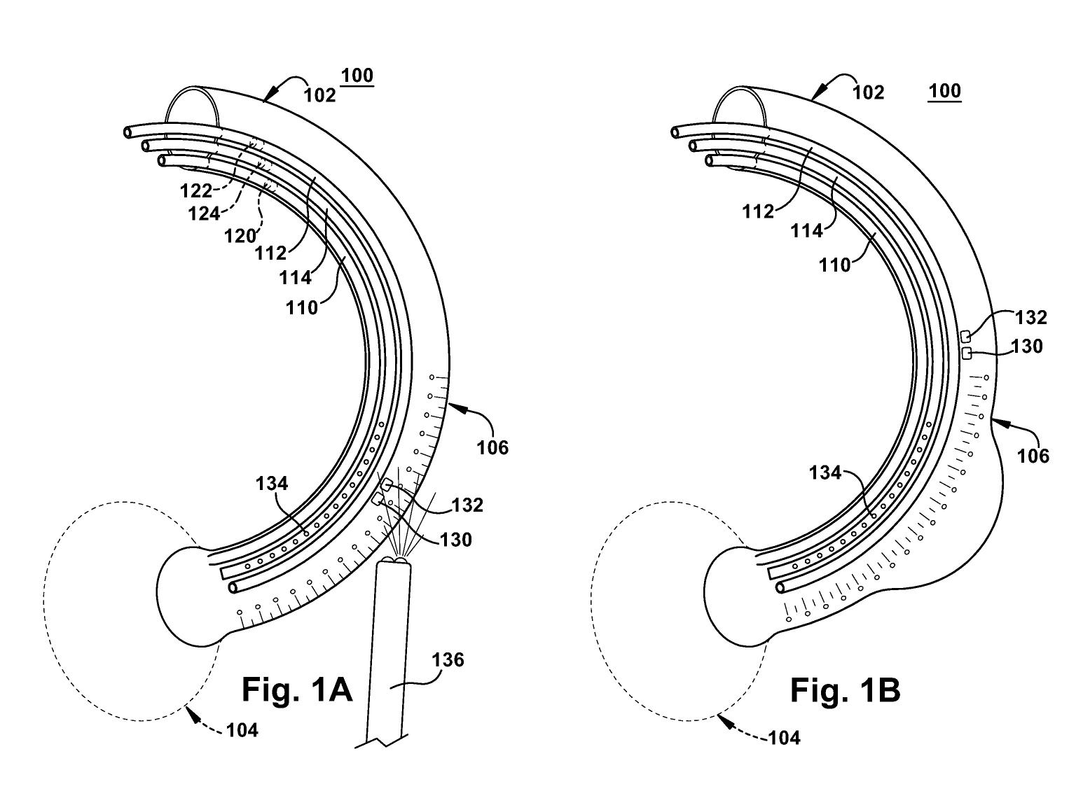

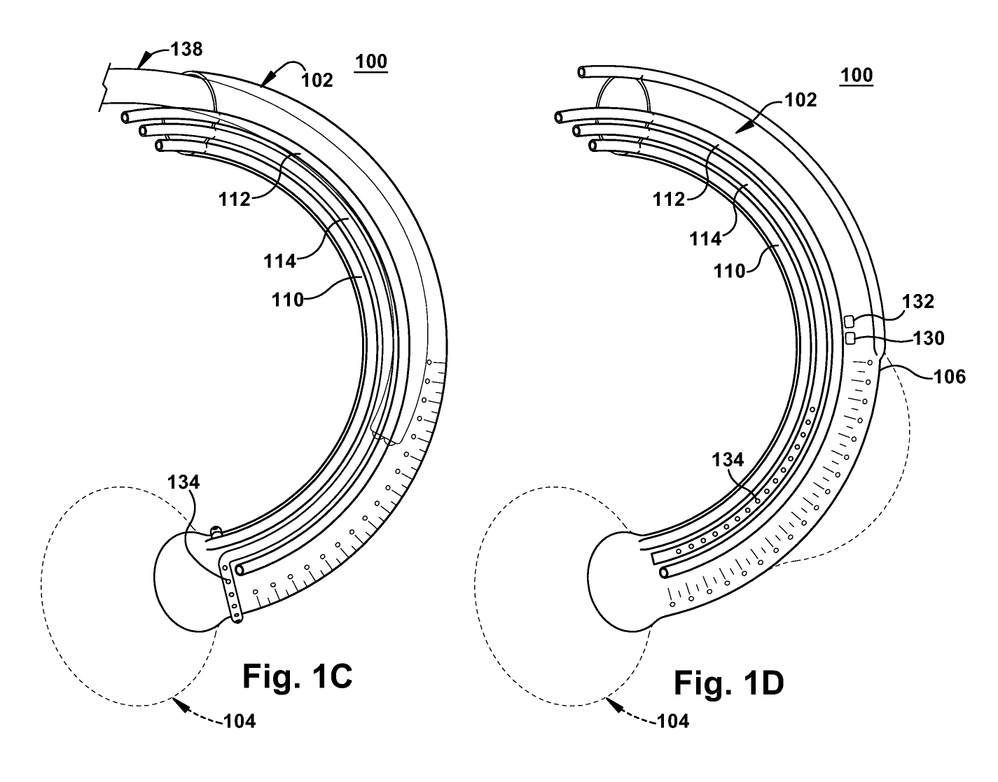

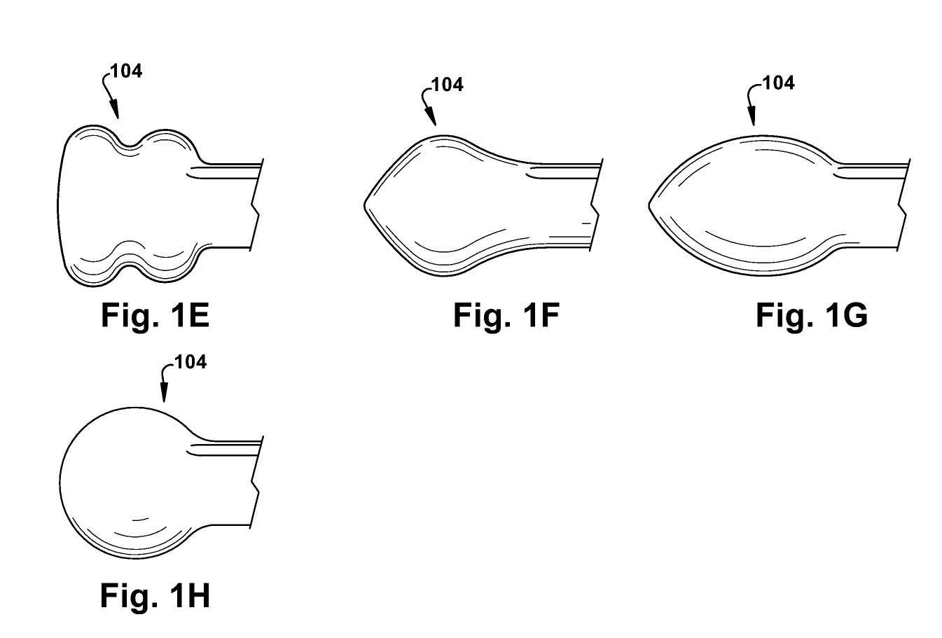

FIG. 1A is an illustration of an exemplary sleeve-tube 100 in accordance with one or more aspects as will be described in more detail herein. The sleeve-tube 100 may comprise a bougie, in the form of tube 102. In one exemplary embodiment, tube 102 may be selectively closed off at one end with an operatively coupled inflatable member, such as balloon 104. According to one aspect, tube 102 may be clear, hollow, and/or comprise standardized markings 106 to enable the surgeon to determine a better estimate of volume, length, width, and/or size of stomach to trim for the operation. For example, the markings may be in inches, centimeters, frenches, liters, and/or other (arbitrary) unit systems. According to another aspect, the standardized markings 106 may begin (e.g. start at zero) where the base of tube 102 meets balloon 104 and run along at least a portion of a length of the tube 102. Alternatively, depending on the balloon 104 shape and/or size, the markings may begin at a number higher than zero to compensate for the thickness of the balloon, for example.

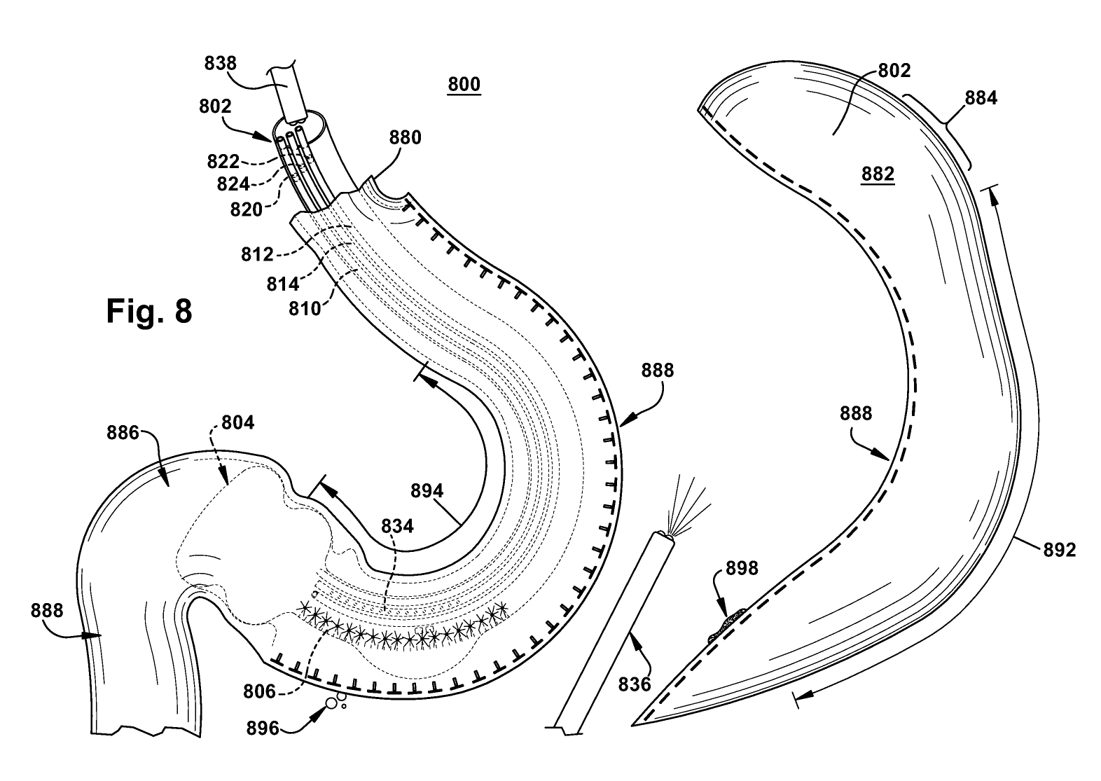

It will be appreciated that standardized markings 106 may be raised in a manner to provide surgeons with additional feedback during the procedure (e.g. slight bumps and/or indentations for visual feedback), for example. Accordingly, tube 102 may comprise (e.g., volcano shaped) bumps running along the length, and the respective bumps could represent one unit. Therefore, these bumps may provide the surgeon with more precise measurement information pertaining to the size of the patient's stomach. Moreover, standardized markings 106 may be configured to emit light in a manner which would provide surgeons visual cues (e.g., as will be discussed in FIG. 8), viewed from the exterior, through the tissue of the stomach, for example. According to one aspect, a surgeon could insert sleeve-tube 100 into a patient's stomach, and laparoscopically 136 view the light markings externally. One of the advantages of internal standardized light markings, among others, is that even though the bougie and/or tube 102 may be contained within the patient's stomach, the standardized markings 106 may shine through the tissue, enabling the surgeon to view the markings 106 from the exterior of the stomach. Therefore, it is believed the surgeon could perform LSG without an internal view of the stomach while maintaining the ability to accurately size the stomach. In one embodiment, tube 102 may be opaque, or a dark color to enhance contrast between the tube 102 and standardized markings 106 (e.g. the tube 102 may be dark and standardized markings 106 may be bright, allowing less light to diffuse through the tube 102, for a higher contrast ratio, making the markings 106 easier to follow).

Turning to FIG. 1B, tube 102 may take the shape and/or size of a typical stomach for a patient, for example. Alternatively, tube 102 may be shaped in a variety of other shapes (e.g., cylindrical) and/or substantially follow a curvature of the stomach. For example, the shape and/or size of tube 102 may be based on factors relating to the patient, such as height, weight, gender, desired weight, and/or health history. FIG. 1B illustrates an exemplary embodiment where tube 102 may be shaped similar to a stomach. One advantage of shaping tube 102 in a stomach like manner is that the extra width of the tube 102 inhibits post surgery kinking of the stomach, for example. In some situations, surgery based on thinner tube shaped bougies may require external lateral support from adjacent organs to inhibit kinking and/or food blockage. That is, a surgeon may stitch a portion of the remaining stomach sleeve to other portions of the body to allow food to pass through without potential blockage. Therefore, shaping tube 102 in the shape of a stomach is believed to potentially mitigate kinking, blockage, preventative surgical measures, and/or additional surgeries.

In one embodiment, the tube 102 may be around two to four feet long, have a radius from approximately twenty eight to sixty frenches, and comprise standardized markings 106 running along at least a portion of the length of the tube. According to one aspect, standardized markings 106 may begin where the balloon 104 meets the tube 102. It will be appreciated that various patients have differing needs, so the attributes, dimensions, length, width, thickness, volume, shape, size, and/or radius of tube 102 and/or balloon 104 may be tailored accordingly for various patients. For example, a taller patient may require a longer tube 102 than a shorter patient. Similarly, a surgeon may choose a smaller volume balloon 104 depending upon the obesity of the patient.

In FIGS. 1A-1D, balloon 104 is depicted in a deflated state (with potential inflation illustrated in phantom), while FIGS. 1E-1H illustrate the balloon in an (at least partially) inflated state, for example. In one exemplary embodiment, the balloon 104 comprises rubber and takes the shape of a dumbbell (as illustrated in FIG. 1E) upon inflation, and may be operatively coupled lengthwise to the tube 102. In another embodiment, illustrated in FIG. 1G, the inflated balloon 104 may be somewhat teardrop shaped and/or shaped like an American football. Yet another possibility would be a spherically shaped balloon 104, as depicted in FIG. 1H. It will be appreciated that balloon 104 may be configured to inflate to form any shape capable of blocking the pylorus and/or separating the lower intestines from the stomach. As an example, upon inflation, balloon 104 may take the shape of a pear (as illustrated in FIG. 1F), an oval, a pyramid, a cone, or a sphere, etc. Depending on the patient, differently shaped balloons 104 may create better separation between the stomach and the pylorus, for example. To this end, if a patient has a large duodenum, a pear shaped balloon or a dumbbell shape may be more efficient than a spherical balloon. To this end, a particular type/shape of balloon may be chosen depending upon the particular patient. It will be appreciated that the balloon 104 may comprise any soft, pliable material (e.g. plastic, silicone, synthetics, and/or other latex free materials) suitable for medical use.

In example FIG. 1A, among others, sleeve-tube 100 comprises secondary channels such as balloon inflation channel 110, stomach inflation channel 112, and applicator channel 114. In one embodiment, balloon inflation channel 110, stomach inflation channel 112, and/or applicator channel 114 may be disposed along at least some of a length of tube 102. It will be appreciated that balloon inflation channel 110 may be configured to inflate and/or deflate balloon 104, and various gases, liquids, and/or solids may be pumped, removed, and/or provided through the balloon inflation channel 110 during the process of inflation and/or deflation. As an example, balloon 104 may be inflated and/or deflated via balloon inflation channel 110 with air and/or water. Stomach inflation channel 112 may be configured to pump and/or provide various gases, liquids, and/or solids into and/or out of the stomach. The stomach inflation channel 112 thus has discharges, opens, exits, etc. (possibly at more than one location) into the stomach (e.g., whereas the balloon inflation channel 110 discharges, opens, exits, etc. into the (interior of the) balloon). In one exemplary embodiment, stomach inflation channel 112 provides a gas, such as air for slight inflation of the stomach, to facilitate stapling. Moreover, stomach inflation channel 112 may be configured to provide and/or pump gas, other fluid(s), etc. into the stomach after the stapling is complete to test for leaks around the exterior portions of the staple line. Therefore, a surgeon may take preemptive measures to prevent leaking if gas, other fluid(s), etc. is noticeably escaping through the stapled tissue.

Applicator channel 114 may be configured to deliver at least one of medicine, hemostats, sealants, glues, powders, gases, liquids, and/or solids mixtures to the interior of the stomach. Thus, like the inflation channel 112, the applicator channel opens (possibly at more than one location) into the stomach. It will be appreciated that balloon inflation channel 110, stomach inflation channel 112, and/or applicator channel 114 may comprise one or more valves to control the flow of substances within the respective channels. For example, applicator channel 114 may comprise applicator valve 124 to control delivery of a substance in a desired/quantifiable manner. Further, stomach inflation valve 122 and balloon inflation valve 120 may be configured to open or close so constant (e.g. externally applied/provided) pressure is not required to maintain inflation. In this way, stomach inflation valve 122 and balloon inflation valve 120 may be configured to facilitate testing for leakage, for example. According to one aspect, balloon 104 may be inflated with air from balloon inflation channel 110, and balloon inflation valve 120 may be closed to see if balloon 104 shrinks (or changes in size) noticeably. According to another aspect, stomach inflation channel 112 may be configured to test the seal between balloon 104 and the pylorus. For example, balloon 104 may be reconfigured to block the pylorus if stomach inflation channel is active and the stomach does not inflate (e.g., indicating that air, etc. is escaping through the pylorus). Further, after the surgeon has performed the stapling, gas may be pumped through stomach inflation channel 112 and stomach inflation valve 122 shut thereafter. Should the surgeon observe a disproportionate amount of gas escaping through the staple lines, then adjustments may be made accordingly (e.g., more staples applied). In one embodiment, the surgeon may inflate the stomach via stomach inflation channel 112 using a dye, liquid, and/or gas to trace leakage through the staple line (e.g., after shutting stomach inflation valve 122). It will be appreciated that balloon inflation channel 110, stomach inflation channel 112, and/or applicator channel 114 be may configured to pump, provide, draw, and/or remove a variety of substances.

It will be appreciated that applicator channel 114 may comprise one or more dispensing heads 134 which may be patterned to dispense in multiple areas. As an example, applicator channel 114 may be setup to dispense along a staple line, along a curve (as illustrated in FIG. 1A), to distribute in a circular (illustrated in FIG. 1C), or a spherical manner. In the illustration of FIG. 1B the dispensing heads 134 may be setup for linear dispersal. For example, if a staple line is anticipated to run in a curved fashion, the dispensing heads 134 could be configured to spray in a curve similar to the staple line. It will be appreciated that tube 102 may rotate within the patient to further facilitate application, for example.

According to one aspect, a surgeon may inflate the stomach using balloon inflation channel 110 for enhanced access to the stomach during the surgery. Balloon inflation channel 110 may be configured to inflate the balloon 104 and block the pylorus, such as during LSG surgery. One of the many advantages, among others, the inflatable balloon 104 provides is the ability to control and/or restrict otherwise free flowing fluids, gases, and/or solids from travelling between organs in the operating area to the small intestine and vice versa. Accordingly, this restriction allows the surgeon to perform post operation leakage tests more effectively than in a situation where the pyloric valve is not blocked, for example.

In yet another embodiment, sleeve-tube 100 may comprise light 130 and/or endoscopic camera 132. As previously mentioned, tube 102 may be clear, allowing light 130 and endoscopic camera 132 to provide the surgeon with an internal view during an LSG procedure, for example. While tube 102 is inside the patient, the surgeon may also view standardized markings 106 through endoscopic camera 132, for example. Further, endoscopic camera 132 (e.g., coupled with light 130) may provide a real time view of the surgery from the interior of the stomach to allow the surgeon to check the progress of the surgery as well as the quality of the procedure. In one exemplary embodiment, if bleeding and/or a blood clot occurs during the LSG procedure, the surgeon may view the stomach internally through the endoscopic camera 132 (e.g., coupled with light 130) to manage the problem prior to proceeding with the next portion of the surgery. It will be appreciated that light 130 and/or endoscopic camera 132 may be optional, and since tube 102 may be hollow, a surgeon may choose to forgo the camera 132 and/or lights 130, or insert an endoscope 138 in through the tube 102 at a later time as illustrated in FIG. 1C. Therefore, a light source 130, endoscopic camera 132, and/or endoscope 138 may be selectively receivable within the hollow portion of tube 102, for example.

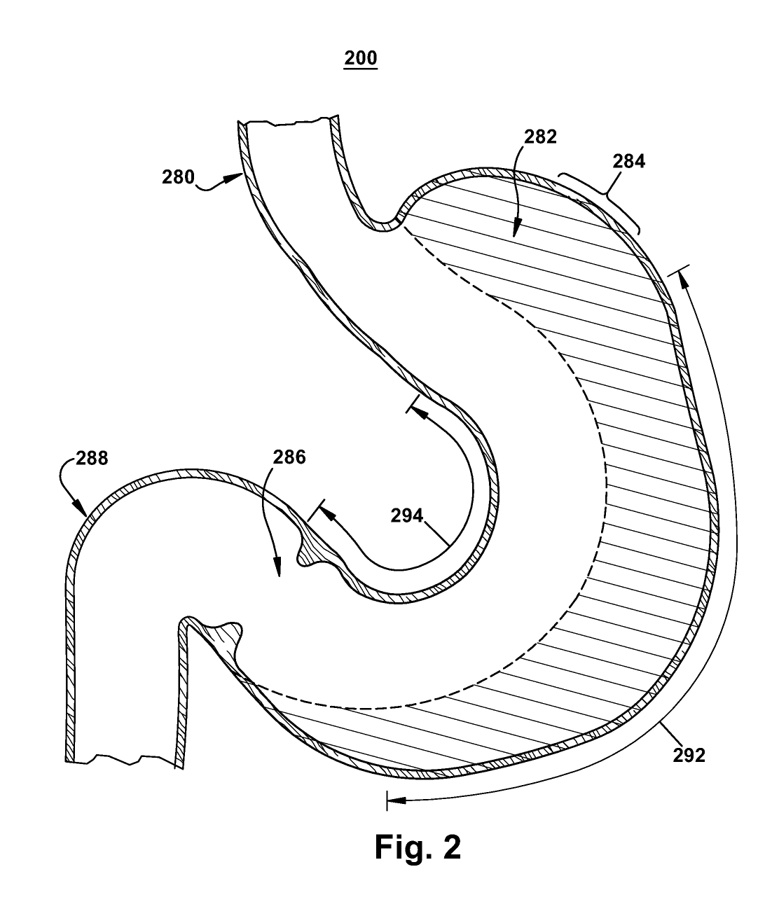

FIG. 2 is an illustration of an example digestive system 200. At 280 food may enter the esophagus, and then the body 284 of the stomach. In the illustrated example, the body 284 of the stomach comprises an excess portion to be dissected 282 (indicated by the shaded region), such as by LSG, for example. After the food is digested, it enters the pylorus 286, and continues through the duodenum to the small intestine 288. The smaller curvature 294 comprises the portion of the stomach which will remain after LSG. Greater curvature 292 is an area where tissue is typically removed during LSG, as surgeons generally trim and/or staple substantially parallel to the greater curvature 292 and/or the lesser curvature. The lesser sac (not shown) is an area generally behind the body 284 of the stomach, and typically may be freed prior to the trimming along the greater curvature 292.

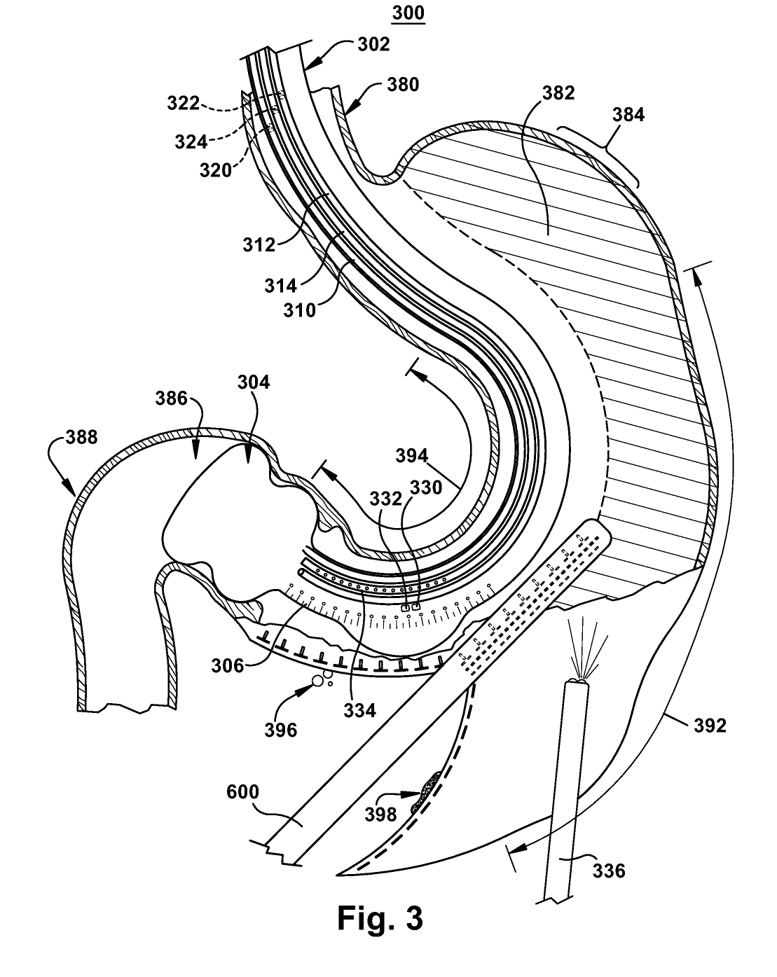

FIG. 3 is an exemplary embodiment of a sleeve-tube device 300 (see, for example one of the sleeve-tube device 100 of FIG. 1) within a digestive system in accordance with one or more aspects described herein. According to one aspect, a surgeon may orogastrically introduce tube 302 through the esophagus 380 and into the body 384 of the stomach. Generally, insertion may cease once a balloon 304 reaches the pylorus 386. In one embodiment, the surgeon inserts the sleeve-tube device 300 while the balloon 304 is in a deflated state. For this example, tube 302 may be clear and/or transparent, and may be constructed out of materials which may be suitable for medical operation purposes. According to another aspect of this example, balloon 304 takes the shape of a dumbbell upon inflation (as illustrated by FIG. 1E) and may be operatively coupled to tube 302 in a lengthwise manner, as illustrated around pylorus 386. According to one aspect, tube 302 comprises the shape, size, and/or radius of a model stomach based on characteristics of the patient (e.g. height, ethnicity, current weight, target weight, gender, and/or average daily caloric intake, etc.). Once the sleeve-tube device 300 is in place, balloon inflation channel 310 may be configured to inflate balloon 304 to block the pyloric valve 386, for example. Balloon inflation valve 320 may be configured to shut and/or seal balloon inflation channel 310 in a manner which maintains the balloon's inflated status while not receiving a constant (e.g. additional) supply of pressure.

According to one aspect, inflation of balloon 304 may control traffic between the body 384 of the stomach and the small intestine 388. It will be appreciated that a light 330 and/or endoscopic camera 332 may be lowered through the hollow section of tube 302 or comprised as a part of the tube 302. For this example, the surgeon may lower an endoscope (e.g. 138 of FIG. 1C) within the hollow portion of the tube 302 and observe therein. Therefore, the surgeon may monitor the interior of the stomach through the endoscope, and also have a view of the exterior of the stomach 384 through a laparoscope 336, for example.

Additionally, the surgeon may choose to inflate the body 384 of the stomach through stomach inflation channel 312 prior to cutting and/or stapling along the greater curvature 392. Stomach inflation valve 322 may operate to control the flow of inflation, and be configured to monitor volume, flow rate, pressure, and/or other statistics related to inflation. For example, stomach inflation valve 322 may monitor an increase and/or decrease in pressure over time and/or a rate at which pressure changes, if at all. That is, stomach inflation valve 322 may be configured to measure a leakage rate, which the surgeon may compare to an expected leakage rate to determine the quality of the pylorus blockage and/or stapling.

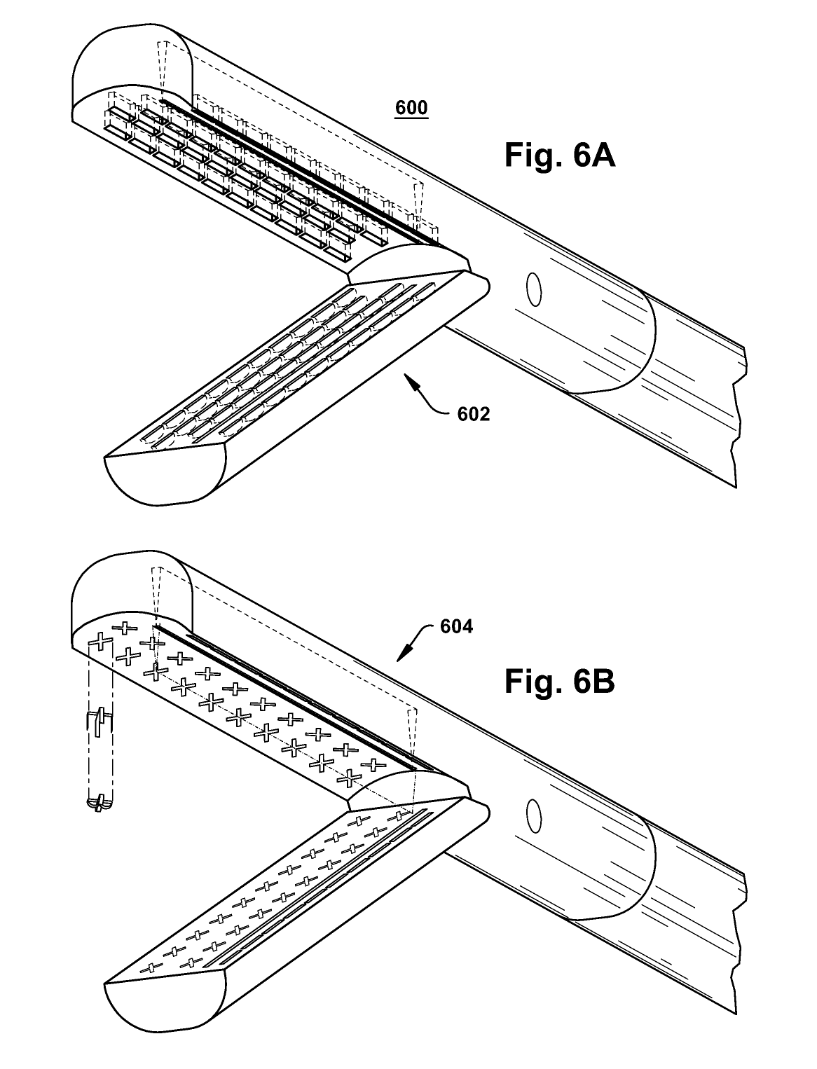

The surgeon may trim and/or staple the excess portion to be dissected 382 using a surgical stapler 600 (as will be discussed in greater detail at least with regard to FIG. 6) to cut, divide, separate, and/or remove the excess portion 382 from the body 384 of the stomach. For example, the surgeon may use standardized markings 306 to determine how much tissue to leave on the body 384 of the stomach. Standardized markings 306 may be in the form of any standardized unit, have a different elevation relative to tube 302, and/or emit lights which shine through the stomach tissue.

Applicator channel 314 may apply medication, hemostats, and/or any other substance to the remaining body 394 of the stomach. In one embodiment, applicator valve 324 may control the amount dispensed while dispensing heads 334 dispense in an appropriate pattern and/or location. For example, the dispensing heads of FIG. 3 dispense in a curved pattern (e.g., compared to the circular pattern of dispensing heads 134 of FIG. 1C).

According to one aspect, after surgical stapler 600 performs the separation along the greater curvature 392, the surgeon may perform a leakage test by way of inflating the body 384 using stomach inflation channel 312. For example, based on the pre-stapling flow rate and/or pressure rate, stomach inflation valve 322 can measure a post-stapling flow rate and/or pressure rate and the surgeon can determine whether the leakage rate is acceptable. To this end, the surgeon may visually observe leaks 396 and 398 (e.g., bubbles, bleeding, etc.) along the greater curvature 392 and/or rely on the statistics provided by the stomach inflation valve 322. Additionally, the surgeon may inflate the body 384 of the stomach with a dye, liquid, and/or gas to aid in identifying leaks 396 and 398 around the stapler line and/or greater curvature 392. For example, stomach inflation channel 312 could pump colored non-toxic liquid and/or gas into the body 384 of the stomach and the colored liquid and/or gas would potentially escape through less securely stapled areas along the greater curvature 392. Therefore, it is believed that a surgeon may be able to re-staple and/or suture such an area accordingly.

Further as illustrated by FIG. 3, the tube 302 of sleeve-tube 300 may be shaped like a stomach, so once the LSG is performed the body 384 may remain shaped like a stomach, for example. One of the advantages of shaping tube 302 like a stomach, among others is that less lateral support may be required from surrounding organs, which potentially mitigates the amount of stitching a surgeon may have to perform. Therefore, it is believed that kinking may be avoided at a point of curvature for the sleeve.

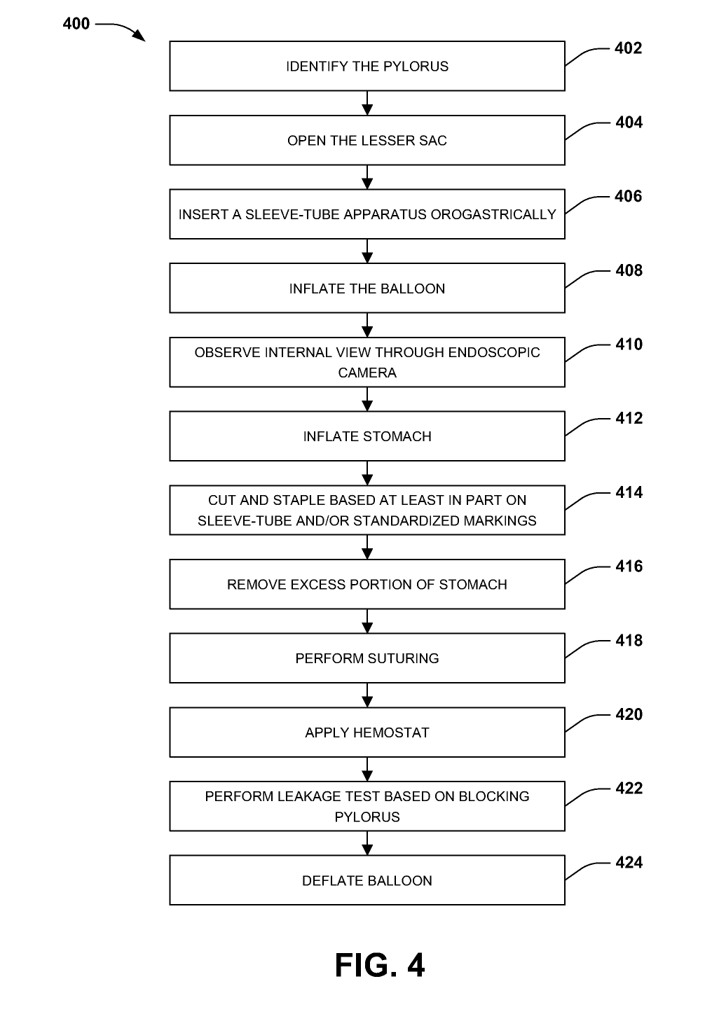

FIG. 4 is a flow diagram of an exemplary method 400 for performing a LSG in accordance with one or more techniques described herein. It will be appreciated that the acts illustrated in FIG. 4 are merely examples and a method described herein may be practiced with more or fewer acts and/or in an order different from that illustrated. At 402, the pylorus may be identified and retraction of the left lobe of the liver may optionally be done. At 404, the lesser sac may be opened while substantially stopping blood flow along the greater curvature of the stomach. For example, a hook may be used to initiate the opening, and an electro-coagulation and/or heat-coagulation device may be used to achieve hemostasis around the area where the blood flow is stopped. At 406, a sleeve-tube bougie may be inserted in an orogastric fashion and while an associated balloon is in a deflated state. It will be appreciated that the dimensions of the sleeve-tube may vary from patient to patient. For example, once the balloon portion of the sleeve-tube has reached the pylorus, the balloon may be inflated at 408 and further insertion of the tube may be halted. Inflating the balloon 408 separates the stomach from the lower intestine and allows leakage testing, such as illustrated in FIGS. 1 and 3, for example. As illustrated in FIG. 1, the sleeve-tube device may take many forms and/or shapes depending, for example, on characteristics of the patient. At 410, the surgeon can observe the progress of the surgery through endoscopic camera and/or lights provided by the sleeve-tube device, or by inserting an endoscope through the hollow/interior portion of the tube. According to one aspect, the surgeon may observe the progress 410 throughout the entire LSG procedure so long as the sleeve-tube is within the patient. At 412, the stomach may be inflated to facilitate the procedure as needed. At 414, the stomach may be cut and/or separated along the greater curvature based at least in part on the sleeve-tube device as a sizing bougie. Standardized markings may be incorporated as part of the cutting procedure 414. For example, a linear stapler may be used to complete the separation of the sleeve and the excess stomach to be removed. At 416, the excess portion of the stomach may be removed. In one embodiment, the surgeon places the excess portion of the stomach in an endobag. Alternatively, the surgeon may remove the excess portion without an endobag, for example. At 418, the surgeon may choose to perform a running suture of the staple line to bury the staple line and inhibit bleeding. The suturing 418 is believed to aid in burying the staples and enhancing the continuity of the staple line. It will be appreciated that the surgeon may leave the sleeve-tube or remove the sleeve-tube at any time, such as after stapling at 414, for example. At 420, a hemostat, medication, and/or any other substance may be applied to the interior of the stomach. For example, distribution may occur via a channel, a pouch, and/or any other means. At 422 a leakage test may be performed, and may be based on visual cues, dye, liquid, and/or gas, rate of change of pressure within the stomach, and/or other statistics related to leakage. At 424, the balloon may be deflated, hemostasis may be further controlled, and the sleeve-tube device may be removed.

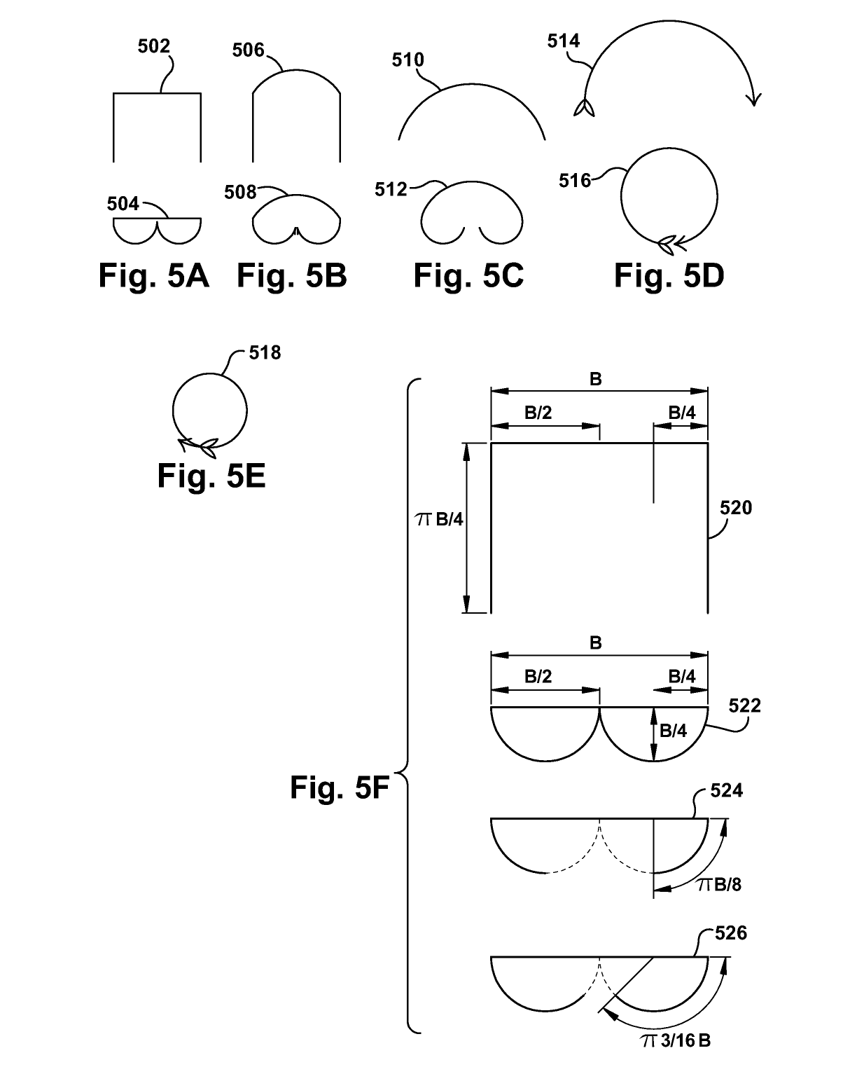

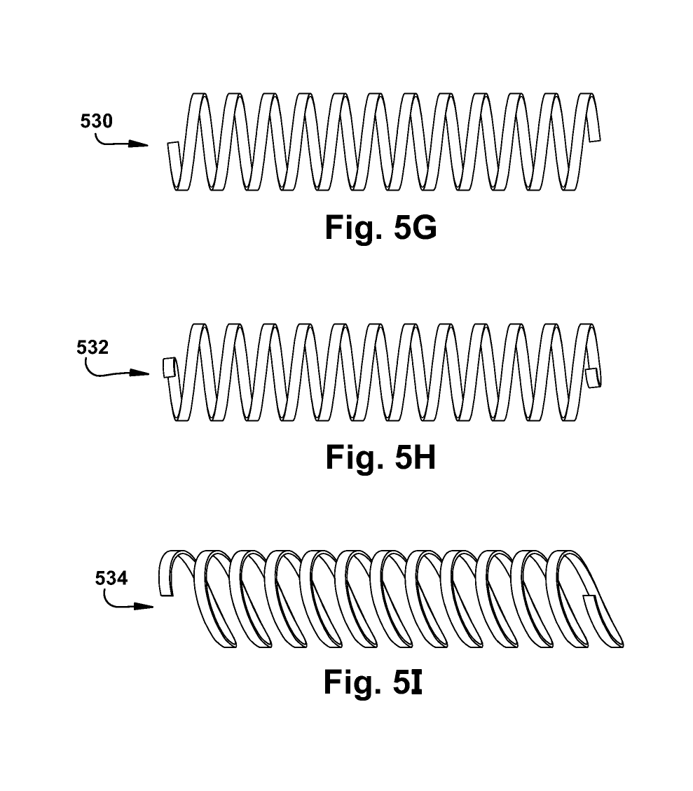

FIG. 5 is an illustration of various (side) views of exemplary surgical staples. In FIG. 5A, a conventional surgical staple is illustrated in both unstapled 502 and stapled 504 forms. In one embodiment, a staple may comprise a bridge and a pair of deformable leg prongs comprising pointed ends, coupled to opposite ends of the bridge, the bridge may be configured to traversely span opposing edges of an incision, and the leg prongs configured to deform to a stapled form. According to one aspect, at stapled state 504, the legs may be deformed in a manner where the pointed ends of the legs may be substantially close to or touching the bridge. According another aspect, the length of the bridge and/or legs may be selected based on the thickness of tissue to be stapled. It will be appreciated that the pointed ends of the legs generally pinch and/or cause pressure points at the stapled tissue, for example. FIGS. 5B-5E illustrate various embodiments of surgical staples configured to reduce trauma to surgically stapled tissue. Upon being deformed into a stapled state, the pointed ends of the legs may be configured in various positions to maintain a substantial gap between the pointed ends and the bridge, for example, while still performing a “stapling” function. It can be appreciated that the gap reduces pressure on tissue sandwiched between the pointed ends and the bridge of the staple, allowing for higher quality (e.g., faster) recovery and less damage to the stapled tissue than conventional staples. The advantages of a substantial gap comprise, among others, the gap replicates natural suturing while potentially achieving a decreased leakage rate, traumatizing less tissue, and providing a sufficient blood supply to the staple line tissue for better healing, for example.

According to one aspect, FIG. 5B illustrates an exemplary surgical staple with an arched bridge and a pair of straight legs with pointed ends. It will be appreciated that the arch to the bridge may be semi-circular, oblong, gateway arch, and/or other arch shapes. At 506 the arched bridge staple is illustrated in an unstapled state, while 508 illustrates the stapled state. FIG. 5C illustrates an exemplary surgical staple with an arched bridge and a pair of arched legs with pointed ends. In one embodiment, the arched curved leg prongs may comprise a curved section prior to being deformed and/or stapled. That is, unstapled staple 510 may be configured to take the shape of a semi-circle and/or other arch shapes, for example. In addition to maintaining a gap between the pointed ends and the arched bridge at 512, the deformable legs may also maintain a horizontal or lateral gap (e.g., substantially parallel to the bridge) between each other. According to another aspect, any staple of FIG. 5 may be configured to maintain a horizontal gap similar to the one depicted in FIG. 5C. In FIG. 5D, one embodiment of a surgical staple is illustrated comprising a bridge, two legs, one leg comprising a pointed end, and the other leg comprising a pointed end acceptor configured to hold the opposing leg prong in place after stapling, for example. Although the bridge and legs of the staple in FIG. 5D may be arched, it will be appreciated that the bridge and/or the legs may be configured to be straight, for example. At 514, the staple is in an unstapled form, while 516 illustrates the stapled form of the staple (e.g., acceptor holding opposing leg prong). In one embodiment, pointed end acceptor may be configured to hold the pointed end in place once the staple deforms to take stapled form 516. According to yet another aspect, upon being stapled 516, the staple may form an oval and/or circular shape. FIG. 5E illustrates yet another aspect of stapled form 518 for unstapled staple 514 (e.g., opposing leg prong slightly pulled through or drawn past acceptor).

According to another aspect in FIG. 5F, a staple is illustrated at 520 with a leg length calculated relative to the length of the bridge (or the horizontal length between the legs for an arched bridge). In this way, the stapled form at 522 may be configured to apply varying amounts of pressure on the stapled tissue based on the length of the legs, for example. Accordingly, if a staple comprises a bridge length of ‘B’ at 522 and the legs form half-circles in the stapled form, then the half-circles may comprise a diameter of B/2 and a radius of B/4. Therefore the half-circle comprise a circumference of pi*B/4. Thus, if the legs were of length pi*B/8, then deforming the legs would create a quarter of a circle as illustrated in FIG. 5F at 524. According to one aspect, the leg prongs may comprise a length shorter than pi*B/4 and longer than pi*B/8 (to yield desired tissue stapling). In one embodiment 526, the legs may be configured to deform to take the shape of ⅝ of a half-circle and may comprise a length of pi*3*B/16. In this embodiment, the curvature of the legs stops at around forty five degrees from full completion of a half-circle. In another exemplary embodiment, the legs may comprise a length anywhere between pi*B/8 and pi*B/4, giving the staple a range between a quarter circle and less than a full half-circle after taking the stapled form. It will be appreciated that the length of the legs and the extent of curvature may be changed based at least in part on the thickness of tissue being stapled. For example, if one type of tissue may be thicker than another type of tissue, then the thicker tissue may take staples configured with longer legs, while maintaining the appropriate ratio between the bridge and the leg lengths. Further, the bridge and leg lengths may be adjusted based at least in part on the type of tissue being stapled. For example, one type of tissue may tend to slip, leak, and/or staple less cooperatively than another type of tissue, so the legs and/or the bridge may be adjusted accordingly.

According to one aspect, FIG. 5G illustrates a spiral shaped staple which may be implanted in a patient to join and/or seal opposing edges of tissue of a patient, for example. According to one aspect, the spiral staple may be injected in a corkscrew like fashion and be configured to traversley span opposing edges of an incision. At 530 the staple is illustrated in unstapled form, while FIG. 5H illustrates the stapled form 532 where the pointed ends of the leg prongs may be deformed to prevent the staple from unwinding. Due to the nature of the spiral shape, less tissue may be traumatized and/or crushed during stapling, which enables better recovery for the patient. For example, the spiral staple replicates small punctures (e.g., more akin to suturing) as opposed to a conventional staple which compresses the tissue. It will be appreciated that the radius of the spiral and the distance between coils may be adjusted from patient to patient, depending on the surgery, tissue thickness, and/or tissue type being stapled. Further, alternative stapled form 534 may compress the coils in a manner which does not traumatize the tissue in the way conventional staples do. For example, the coils of the spiral may compress similar to falling dominos, as illustrated at 534 of FIG. 5I (e.g., the coils fall or lay upon one another). Further, the staples of 530, 532, and/or 534 may be fed from a spool and not require reloading of a new cartridge such as the one depicted in FIG. 6B at 604, for example. Additionally, staples 530, 532, and/or 534 may be fashioned into and/or cut to length, such as by the stapler 600 (e.g. to form individual staples such as at least one of the staples of FIGS. 5B-5F).

It will be appreciated that any of the staples illustrated in FIG. 5 may be of varying thicknesses and crafted from materials acceptable for surgery. For example, the staples could comprise plastic, titanium, and/or other materials. Further, the length and/or shape of the bridge and/or the legs for the respective staples may vary depending on the surgical application, the thickness of the tissue, and/or the type of tissue, for example.

FIGS. 6A and 6B are illustrations of exemplary surgical staplers in accordance with one or more aspects described herein. At 600 a surgical stapler is illustrated and configured to fire various types and sizes of staples. According to one aspect, the stapler may comprise a jaw comprising a top 604 and bottom 602 cartridge, at least one of the cartridges movable with respect the other cartridge between an open and a closed position. According to another aspect, at least one of the cartridges is interchangeable and/or removable. In one embodiment, the stapler applies substantially parallel rows of surgical staples to tissue while concurrently forming an incision between the rows of staples when the jaw of the stapler closes. It will be appreciated that the surgical stapler may be configured for use during an endoscopic, laparoscopic, and/or open surgical procedure. Further, the surgical stapler may be one of a linear, curved, and/or any other pattern. The device can be loaded with staples and configured to apply staples based at least in part on a pattern of an interchangeable staple cartridge 604. In an exemplary embodiment illustrated by FIG. 6B, the surgical stapler staples concurrently while cutting along a segment of tissue, where tissue is cut with blade (illustrated in phantom in a first hidden position corresponding to an open jaw position and a second extended position corresponding to a closed jaw position) and stapled when a jaw of the stapler is closed, bringing top and bottom cartridges in closer proximity to one another. For example a cutting blade may extend from a hidden position (e.g. the blade within the cartridge) to a cutting position (e.g. illustrated in a protruding or emanating position) upon a closing of the jaws of the stapler. Additionally, when the jaws are closed, staples may be emanated from a staple cartridge in a concurrent, overlapping fashion (illustrated in phantom, in both unstapled and/or stapled forms) to form a crossing pattern and/or an “x”, in the illustrated example, for additional security along a staple line. In another example, different cartridges may comprise a differing number of rows and/or patterns, as will be discussed herein. Further, the surgical stapler 600 may be configured to fire staples in a manner which maintains a gap between the pointed ends of the legs and the bridge of the staple. By way of example, surgical stapler 600 may be configured to fire any of the staples illustrated in FIG. 5. However, it will be appreciated that surgical stapler 600 may be configured to fire other embodiments of staples, and is not limited to those illustrated in FIG. 5.

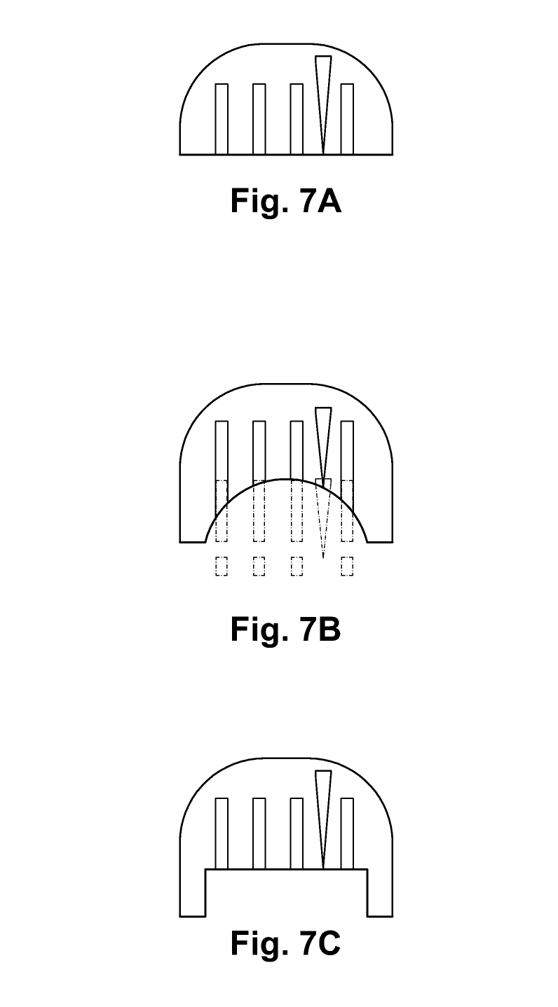

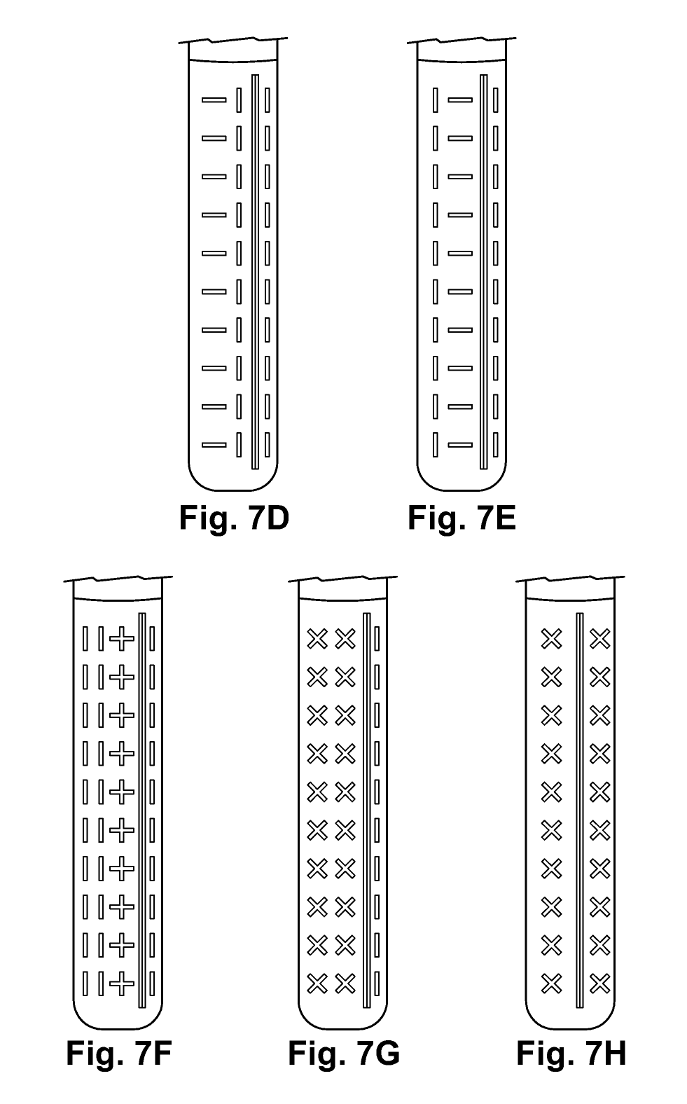

FIG. 7 is an illustration of exemplary surgical stapler cartridges in accordance with one or more aspects described herein. It will be appreciated that the cartridges of FIG. 7 may be interchangeable and/or swapped with at least one of the (top) cartridges of surgical stapler 600 of FIG. 6. In FIGS. 7A, 7B, and 7C staples emanate from the rectangular regions while tissue is cut by the blade (illustrated by the triangular wedge shaped element) when the jaws of a stapler (within which the cartridge is installed) are closed. In one embodiment, when the jaw (e.g. of stapler 600 of FIG. 6) closes, staples are forced out of the cartridge and the blade drops to cut tissue (e.g., via one or more springs in the stapler). FIG. 7A illustrates a front (cross-sectional) view of a conventional surgical stapler cartridge (e.g., top or upper cartridge in FIGS. 6A and 6B). It can be appreciated that due to the flat configuration of the part of the cartridge that contact the tissue being stapled (e.g., when a stapler jaw is closed), most if not all of the tissue may be crushed and/or traumatized. Therefore, the tissue healing may be less than optimal. According to one aspect, FIG. 7B illustrates an arched staple cartridge, which maintains at least a semi-circular gap while the jaw of the stapler is in the closed position for firing/stapling so that less, if any, pressure is imposed upon tissue being stapled. Further, FIG. 7B illustrates a hidden position of a cutting blade (illustrated by the triangular wedge shaped element) as well as an extended or cutting position of the blade (illustrated in phantom) for an arched staple cartridge. Additionally, FIG. 7B illustrates staples emanating from the arched staple cartridge in an unstapled form (e.g. partially emanating from the cartridge) and a stapled form (e.g. fully ejected from the cartridge). It will be appreciated that, in one example, the staples are deformed or placed into their respective stapled forms based at least in part upon encountering a corresponding (recessed) location in a bottom jaw of the stapler when the stapler is closed and the jaws thereof are brought closer to one another. It will also be appreciated that the orientation illustrated in FIG. B (and FIGS. 7A and 7B) provides a view into one side of a staple (e.g., 90 degrees relative to the views illustrated in FIGS. 5A-5F). That is, merely one leg of the staple may be visible, but generally not the bridge or other leg of the staple (e.g., as the other leg would be behind and thus obscured by the visible leg of the staple), particularly before the staple is deformed into its stapled configuration (e.g., as some of the bridge may “arch-up” and thus be visible upon deformation). Yet another aspect is illustrated in FIG. 7C where a square shaped cavity is left between the jaws of the closed stapler. The height of the gap in FIGS. 7B and 7C may be based at least in part on the thickness of the tissue being stapled. For example, if stomach tissue is approximately five millimeters thick, then the staple cartridge could be configured to leave a gap sufficient to staple but not crush the tissue when the stapler jaw is in a closed position.

According to another aspect illustrated by FIGS. 7D-7H, the staple cartridges may comprise different staple patterns. In FIG. 7D, the cutting blade may be located slightly right of center of the cartridge while one side comprises a row of staples running substantially parallel with the cutting blade. The opposite side of the cutting blade comprises two rows of staples, the row closest to the cutting blade substantially parallel to the cutting blade, and the second row substantially perpendicular to the cutting blade. FIG. 7E illustrates a counterpart to FIG. 7D, where one side comprises a row of staples running substantially perpendicular to the cutting blade, while the opposite side comprises two rows of staples, the row closest to the cutting blade substantially perpendicular to the cutting blade, and the second row substantially parallel to the cutting blade. In another aspect, the staples may be aligned an angle to the cutting blade or placed in an overlapping fashion, as illustrated by FIGS. 7F-7H. In one embodiment, the staple on top of the overlapping pattern may be configured to comprise different dimensions than the staple under the top staple. Therefore, the staple on top may comprise a longer bridge length, and/or a longer leg length than the bottom staple. It should be appreciated that the staple patterns may comprise any number of rows of staples on either side of the cutting blade, and the respective rows may be overlapping, substantially perpendicular, substantially parallel, and/or at an angle (e.g. other than ninety degrees) independent of other rows relative to the cutting blade. It can be appreciated that having fewer staples to one side of the blade mitigates waste, among other things. For example, the blade may be used to dissect a portion of a stomach (e.g., during LSG) and the dissected portion of the stomach that is going to be discarded may merely comprise a single row of staples whereas the portion of the stomach that remains in the patient may comprise more staples as it is more important to mitigate leakage, for example, in the patent than in a portion of the stomach that is going to be discarded. Accordingly, unlike conventional cartridges which generally apply an even number (and same pattern) of staples to tissue on either side of an incised area, the subject matter herein results in fewer (expensive surgical) staples being discarded and generally more effective stapling of remaining non-dissected tissue.

FIG. 8 is an illustration of surgical staple lines within example digestive system in accordance with one or more aspects described herein. The greater curvature 892 comprises excess portion to be removed 882, and the body 894 of the stomach may remain. The surgeon may opt to insert a sizing bougie or a sleeve-tube device as diagrammed by FIG. 1. Accordingly, while cutting the surgical stapler may fire one or more staple lines 888 along the excess portion to be removed 882 and one or more staple lines 888 along the remaining body 894 of the stomach. It will be appreciated that the staple lines may be patterned after any one of the cartridges illustrated in FIG. 7 (e.g., 7D in the illustrated example). However, it will be appreciated that the surgical stapler may be configured to fire other patterns, and is not limited to those illustrated in FIG. 7. Further, tube 802 may comprise standardized markings 806, which may be configured to shine light through tissue of stomach 894. Therefore, this would provide the surgeon with visual feedback based on a standardized sizing bougie. Also, hemostat and/or other material(s) may be selectively applied at desired locations.

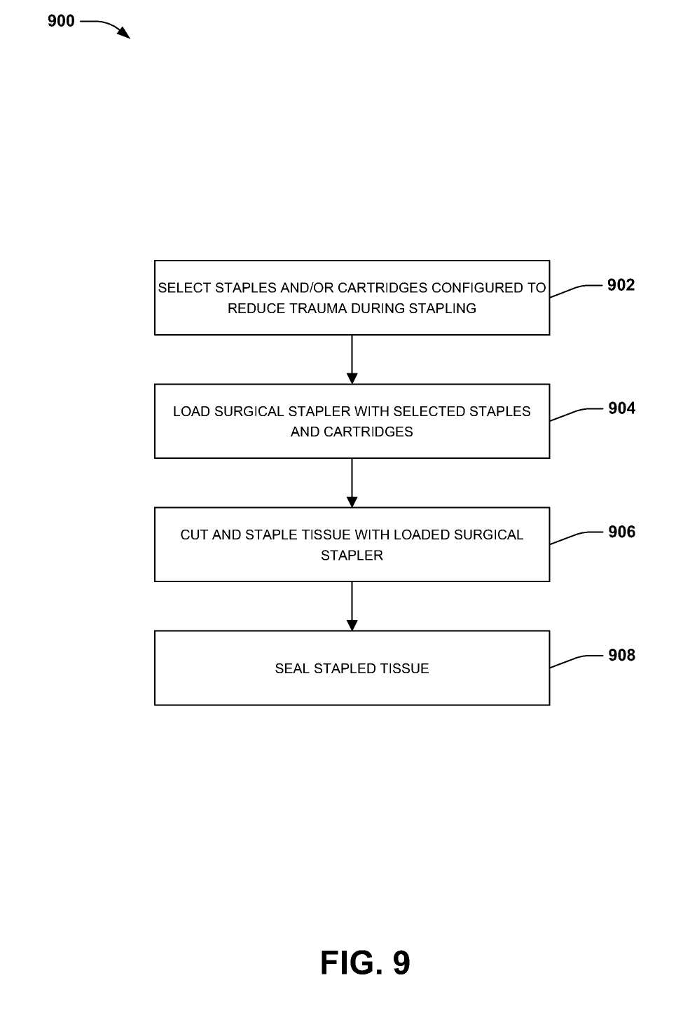

FIG. 9 is a flow diagram illustrating an example embodiment where one or more techniques described herein may be implemented. It will be appreciated that the acts illustrated in FIG. 9 are merely examples and the method described herein may be practiced with more or fewer acts and/or in an order different from that illustrated. At 902 one or more sets of staples and/or staple cartridges may be selected where the cartridges and/or the staples may be configured to reduce trauma to stapled tissue. The selection at 902 may be based at least in part on at least one of a type of surgery being performed, a thickness of tissue being stapled, and/or a type of tissue being stapled, etc. Further, the staples of 902 may be configured to maintain at least a gap between the pointed ends of the legs and the bridge upon deforming to the stapled state. The staple cartridge of 902 may be configured to maintain a gap while the jaws of the surgical stapler may be in a closed position. In this way, the staple cartridges may be configured to reduce the amount of surface area which clamps down on tissue while the jaw of the surgical stapler is in the closed position. At 904 a surgical stapler may be loaded with the selected cartridges and staples of 902. At 906, the surgical stapler cuts and staples tissue (e.g., in a relatively less traumatizing manner), leaving a pattern based on the selected cartridges. At 908, the tissue may be sealed (e.g., by applying hemostat and/or other material(s)).

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing at least some of one or more portions of at least one of the claims.

Moreover, the word “exemplary” is used herein to mean serving as an example, instance, or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims may generally be construed to mean “one or more” unless specified otherwise or clear from context to be directed to a singular form. Further, at least one of A and B and/or the like generally means A or B, or both A and B.

Although the disclosure has been shown and described with respect to one or more implementations, equivalent alterations and modifications may occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure. In addition, while a particular feature of the disclosure may have been disclosed with respect to only one of several implementations, such feature may be combined with one or more other features of the other implementations as may be desired and advantageous for any given or particular application. Furthermore, to the extent that the terms “includes”, “having”, “has”, “with”, or variants thereof are used in either the detailed description or the claims, such terms are intended to be inclusive in a manner similar to the term “comprising.”

What is claimed is:

1. A device configured to promote standardizing tissue resection during sleeve gastrectomy comprising:
a tube comprising one or more markings along at least some of a length of the tube;
a balloon operatively coupled to one end of the tube, the balloon configured to block a surrounding orifice upon inflation; and
a balloon inflation mechanism disposed along at least some of the length of the tube and configured to at least one of inflate or deflate the balloon.

2. The device of claim 1, comprising one or more secondary inflation channels disposed along at least some of the length of the tube and configured to at least one of provide or remove at least one of a gas, a liquid, or a solid to an area exterior to the tube.

3. The device of claim 1, the balloon inflation mechanism comprising a balloon inflation channel.

4. The device of claim 3, the balloon inflation channel configured to inflate the balloon based at least in part on providing at least one of a gas, a liquid, or a solid.

5. The device of claim 2, the tube configured to receive at least one of a light source or a video camera.

6. The device of claim 2, the markings comprising units, the units comprising at least one of a metric unit, an English measurement unit, a French, a centimeter, or an inch.

7. The device of claim 2, comprising one or more applicator channels disposed along at least some of the length of the tube and configured to provide at least one of an adhesive, a medicine, or a hemostat to an area exterior to the tube.

8. A surgical staple configured to seal opposing edges of an incision in tissue of a patient, comprising:
a bridge configured to traversley span the opposing edges of the incision; and
a pair of leg prongs coupled to opposite ends of the bridge, the pair comprising a first leg prong coupled to a first end of the bridge and a second leg prong coupled to a second end of the bridge, the first leg prong having a first pointed end and the second leg prong having a second pointed end, the first leg prong and the second leg prong configured to deform in a manner which maintains a substantial gap between at least one of the first pointed end and the bridge upon stapling or the second pointed end and the bridge upon stapling.

9. The surgical staple of claim 8, the bridge comprising a bridge length based at least in part on a thickness of the tissue.

10. The surgical staple of claim 8, at least one of the first leg prong or the second leg prong comprising a leg length based at least in part on a thickness of the tissue.

11. The surgical staple of claim 8, the bridge comprising an arched section.

12. The surgical staple of claim 8, at least one of the first leg prong or the second leg prong comprising a curved section prior to being deformed.

13. The surgical staple of claim 8, at least one of the first leg prong or the second leg prong comprising a leg length:
shorter than pi*B/4, where B represents a length of the bridge; and
longer than pi*B/8, where B represents the length of the bridge.

14. The surgical staple of claim 8, at least one of the first leg prong or the second leg prong comprising a leg length of pi*3*B/16, where B represents a length of the bridge.

15. The surgical staple of claim 8, the first leg prong comprising a leg prong acceptor configured to hold the second leg prong in place upon stapling.

16. A trauma decreasing surgical stapler, comprising:
a jaw comprising a top cartridge and a bottom cartridge, at least one of the top cartridge removable and movable with respect to the bottom cartridge between an open position and a closed position for engaging tissue or the bottom cartridge removable and movable with respect to the top cartridge between the open position and the closed position for engaging the tissue, at least one of the top cartridge or the bottom cartridge arched in a manner which maintains at least a semi-circular gap between the top cartridge and the bottom cartridge while the jaw is in the closed position; and
a cutting blade configured to slice the tissue when the jaw engages into the closed position, at least one of the top cartridge or the bottom cartridge configured to implant at least one row of staples into the tissue to a first side of the cutting blade and to a second side of the cutting blade.

17. The stapler of claim 16, the gap comprising a height based at least in part on at least one of a type or a thickness of the tissue.

18. The stapler of claim 16, at least one of the top cartridge or the bottom cartridge configured to implant a staple into the tissue at an angle other than 90 degrees relative to a side of the cutting blade.

19. The stapler of claim 16, at least one of the top cartridge or the bottom cartridge configured to implant a first staple into the tissue substantially parallel to the cutting blade and a second staple into the tissue substantially perpendicular to the cutting blade.

20. The stapler of claim 19, the first staple and the second staple implanted to a same side of the cutting blade.
